(19) United States
(12) Patent Application Publication
(10) Pub. No.: US 2024/0053239 A1
(43) Pub. Date: Feb. 15, 2024

(54) SYSTEM FOR MEASURING THE MECHANICAL PROPERTIES OF A SKIN SAMPLE

(71) Applicants: UNIVERSITE DE PAU ET DES PAYS DE L'ADOUR, PAU (FR); CENTRE NATIONAL DE LA RECHERCHE SCIENTIFIQUE - CNRS, Paris (FR)

(72) Inventors: Christophe DERAIL, CESCAU (FR); Francis EHRENFELD, MORLAAS (FR); Anthony LAFFORE, ASSAT (FR); Corinne NARDIN, BARINQUE (FR); Bastien BLANCHARD, UZOS (FR)

(21) Appl. No.: 18/258,089

(22) PCT Filed: Dec. 17, 2021
(86) PCT No.: PCT/FR2021/052389

§ 371 Date: Jun. 16, 2023

(30) Foreign Application Priority Data
Dec. 19, 2020 (FR) FR2013764

(51) Int. Cl. G01N 3/08; G01N 33/483; G01N 3/06

(52) U.S. Cl. CPC G01N 3/08; G01N 33/4833; G01N 3/06

(57) ABSTRACT

The present disclosure relates to a system (10) for measuring the mechanical properties of a skin sample (3) ex vivo or in vitro, comprising a measuring device comprising at least one mechanical stress module (20, 40) capable of applying a tensile force to the skin in a direction parallel to the surface of the skin sample (3), the at least one mechanical stress module (20, 40) comprising: —a traction means (30, 50) which is translatably movable in a direction parallel to the surface of the skin sample (3); — a translating arm (21, 41) connected, on the one hand, to the traction means (30, 50) and, on the other hand, to an axial displacement means; — one end of the traction means being provided with an attachment head (31, 51) capable of being attached to a region of the skin sample (3) so as to cause deformation of the skin sample by axially displacing the region of the skin sample— a control unit (202) configured to control the displacement means according to a stress frequency of between 0.1 mHz and 1 Hz, and— a calculation unit (203) configured to receive the signals transmitted by the measuring device and calculate the mechanical properties of the skin from the signals.

### TECHNICAL FIELD

The present disclosure relates to the field of measurements of the mechanical properties of a human or animal skin sample.

More particularly, it relates to devices for measuring the mechanical properties of a skin sample ex vivo or in vitro, deposited and held fixed on a nutritional medium.

### PRIOR ART

Skin has a complex multilayer structure, stratified with three main layers from the surface inwards: the epidermis, dermis and hypodermis. The structural complexity of the skin gives it mechanical properties: anisotropy, elastic behavior, viscoelastic behavior, heterogeneity. The skin can thus be seen as a complex material, the mechanical response of which depends on a large number of factors specific to the person and their living environment: age, sex, health, diet, environment, and region of the human body.

The study of the mechanical properties of the skin in response to a mechanical stress in vivo provides a certain amount of information of primary importance in many fields of cosmetics, surgery and medicine. It is thus known to measure the mechanical properties of the skin in order to evaluate, for example, the state of healthy skin or the changes induced by external products applied to a region of interest of the skin.

Among the mechanical stresses to which the skin is subjected in order to measure the mechanical properties, an important category of mechanical stresses to be applied are tensile stresses in the plane of the skin. The mechanical responses of the stressed skin are then measured by various sensors.

Currently, mechanical stress devices exist which enable traction to be exerted in the plane of the skin.

An example is the extensometer, which can carry out uniaxial traction tests in vivo, by attaching two pads on the skin and moving them in opposite directions. The pads are bonded on the skin in vivo. The bonding is performed with double-sided adhesive strips or with suitable glues.

However, this technique has some disadvantages. Specifically, the use of strong adhesives to obtain an effective anchoring point can, for example, damage the surface layer of the skin when the pads are removed. Hence, it is difficult to recommend this technique for performing tests carried out at the same location on the skin in vivo in order to evaluate the change in mechanical properties over time.

The adhesive strips are less invasive and better tolerated by the patient's skin. However, they do not allow sufficiently strong anchoring points to be obtained, thus giving rise to a risk of a displacement between the pad in contact with the skin and the measurement region, thus causing erroneous measurements.

Hence, current devices are not very accurate for monitoring the change in the mechanical behavior of a given region of the skin in vivo over time.

Another disadvantage is that, between two tests, spaced apart in time, in the event of monitoring over several days, for example, of the skin in vivo, the pads must be able to be removed between two tests and repositioned, which can induce inaccuracies in the location of the studied region which can impact on the reproducibility of the test results.

Another disadvantage is that current devices are mainly developed for mechanical characterization of the skin in vivo. However, once sampled, the skin loses its mechanical properties over time. Also, it is not currently possible to correctly evaluate the change in mechanical properties over time in a skin in vitro. However, in the case of mechanical characterization of the skin in vivo, the devices and methods must be non-invasive, which leads to limits in the study of the mechanical behavior. In the study of the non-linearity of the force-displacement relation, for example, it is not possible to study the phase corresponding to the rupture phase in the case of characterization of the skin in vivo.

The invention proposes to overcome these disadvantages.

Thus, it is sought to solve the problems posed by the prior art by developing a measuring device capable of evaluating the mechanical properties of the skin ex vivo, which is accurate, cost-effective and easy to use.

Another aim of the invention is to provide a measuring device capable of carrying out measurements in various possible directions in the plane of the skin.

Another aim of the invention is to provide a measuring device capable of producing mechanical traction stresses parallel to the surface of the skin ex vivo, the mechanical properties of which are maintained over time, and of performing reproducible measurements of the mechanical properties.

### SUMMARY

For this purpose, the object of the present disclosure is a system for measuring the mechanical properties of a skin sample ex vivo or in vitro, comprising a measuring device comprising at least one mechanical stress module capable of applying a tensile force to the skin in a direction parallel to the surface of the skin sample, said at least one mechanical stress module comprising:
a traction means which is translatably movable in a direction parallel to the surface of the skin sample;
a translating arm connected, on the one hand, to the traction means and, on the other hand, to an axial displacement means capable of displacing the traction means;
one end of said traction means being provided with an attachment head capable of being attached to a region of the skin sample so as to cause deformation of the skin sample by axially displacing said region of the skin sample
a control unit configured to control the displacement means, and
a calculation unit configured to receive the signals transmitted by the measuring device and to calculate the mechanical properties of the skin from said signals.

According to one embodiment of the invention, a plurality of mechanical stress modules are arranged around a center of the device and configured to each apply a tensile force in a radial direction parallel to the surface of the skin sample, and the axial displacement means and the translating arms are aligned in pairs so as to displace two traction means along a common displacement axis.

According to one embodiment of the invention, the aligned axial displacement means are synchronized so as to simultaneously displace two traction means along the common axis.

According to one embodiment of the invention, the axial displacement means comprises a piezoelectric nano-positioning table, one end of the translating arm being attached on a moving part of the piezoelectric nano-positioning table.

According to one embodiment of the invention, each stress module further comprises a manual micrometric displacement table configured to manually adjust the position of the translating arm along one of the axes of displacement.

The piezoelectric nano-positioning table and the micrometric displacement table are preferably arranged with respect to one another so as to have the same axis of displacement.

The features disclosed in the following paragraphs can, optionally, be implemented independently of one another or in combination with one another:

The attachment head is in the form of a rod provided with a thread capable of engaging in the thickness of the skin sample to produce a point of attachment in the skin sample.

The attachment head is in the form of a straight cylindrical body, one of the bases of the straight cylindrical body being provided with a layer of adhesive to attach the attachment head to the surface of the skin sample.

The traction means comprises a cylindrical attachment body intended to be received in a recess produced in one end of the translating arm and locked in position using a clamping element.

According to one exemplary embodiment of the invention, the measuring device further comprises at least one tensile force sensor capable of measuring the tensile force applied by a traction means.

According to another exemplary embodiment of the invention, the measuring device further comprises at least one position measurement sensor capable of measuring the position of a translating arm during its displacement.

According to another exemplary embodiment of the invention, the measuring device further comprises at least one imaging means configured to observe the region of deformation of the skin sample caused by the displacement of the attachment heads, the optical axis of said imaging means being oriented in a direction normal to the surface of the skin sample.

### BRIEF DESCRIPTION OF THE DRAWINGS

Further features, details and advantages will emerge from reading the following detailed description and analyzing the appended drawings, in which:

FIG. 1 shows a view from above of a device for measuring the mechanical properties of a skin sample according to one embodiment;

FIG. 2 shows a sectional view of the device of FIG. 1 along the axis (BB′);

FIG. 3 shows a perspective view of the device of FIG. 1;

FIG. 4 shows an enlarged perspective view of the region (B) of FIG. 3;

FIG. 5 shows an enlarged view of a region (A) of FIG. 2;

FIG. 6 shows another view from below of FIG. 4;

FIG. 7 shows a perspective view of a traction means according to two exemplary embodiments of the invention;

FIG. 8 schematically shows a sectional view showing two attachment heads in an attachment configuration in the skin;

FIG. 9 schematically shows the translation of one of the two attachment heads of FIG. 8 in a direction parallel to the surface of the skin, causing a deformation of the region of attachment of the skin by axial displacement;

FIG. 10 shows a system for measuring mechanical properties, comprising the measuring device according to one embodiment of the invention connected to a control unit and to a calculation unit.

FIG. 11 shows the evolution of the complex modulus measured on a sample of pigskin as a function of the stress frequency.

### DESCRIPTION OF THE EMBODIMENTS

In the context of the present disclosure, “mechanical properties” means the physical parameters which can be defined from the deformation of the skin subject to a mechanical stress. Indeed, by analyzing the mechanical responses to the imposed deformation, it is possible to demonstrate the elastic, viscoelastic and plastic properties of the skin.

When traction is exerted on the skin, there is an extension of the skin due to the dermal elastic networks. When the force ceases, the skin retracts with an elastic return to its initial state. In the case of large extension, the deformation is plastic and irreversible.

In the context of the present disclosure, “ex vivo skin sample” means a skin sample taken from a living body and which is kept alive by a nutritional solution, throughout the entire duration of the measurement of the mechanical properties. Consequently, the ex vivo skin sample normally functions as in vivo skin for a determined duration.

In the context of the present invention, “in vitro skin sample” means a synthetic skin sample produced in the laboratory. However, the studies on in vitro skin cannot be used to characterize the natural functioning of skin which is still living.

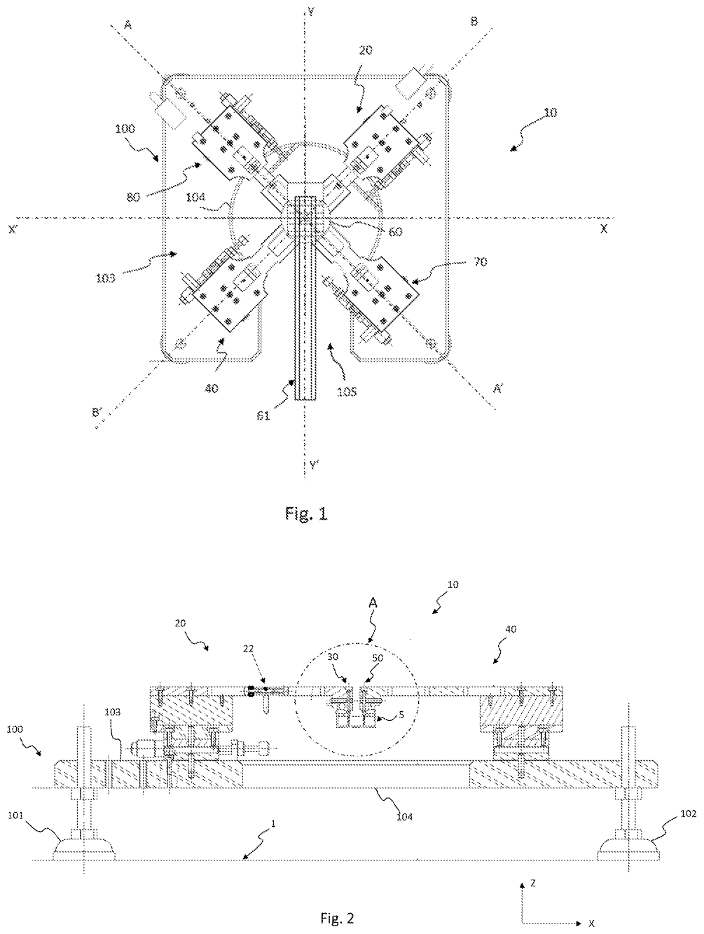

FIG. 1 schematically shows a view from above of the device 10 for measuring the mechanical properties of a skin sample ex vivo or in vitro according to one possible embodiment.

The device of the present disclosure can be used on any type of skin sample. The device of the present disclosure was designed, in particular, for the purpose of characterizing mechanical properties of a skin sample ex vivo held in a fixed position on a nutritional medium which enables the mechanical properties of the skin sample to be maintained for a duration of at least seven days. The technique of keeping an ex vivo skin sample alive is described in document WO2013164436.

The measuring device of FIG. 1 comprises four mechanical stress modules 20, 40, 70, 80 which are each able to apply traction in a direction parallel to the surface of the skin, thus enabling at least four traction tests to be exerted simultaneously in the plane of the skin.

According to one embodiment of the invention, the mechanical stress modules function as pairs. In FIG. 1, module 20 and module 40 are arranged facing one another and exert tractions along a common axis (BB′) in opposite directions. Similarly, the mechanical stress modules 70 and 80 are arranged facing one another and exert tractions along a common axis (AA′) in opposite directions. The axes (AA′) and (BB′) are parallel to the surface of the skin.

The four mechanical stress modules 20, 40, 70 and 80 are supported by a frame 100 intended to be placed and stabilized on a horizontal surface of a table, for example. The frame comprises a base 103 forming a substantially horizontal surface which extends in a horizontal plane (XY). The center of the base 103 is provided with a substantially circular opening 104. The two axes (AA′) and (BB′) are secants at a point located substantially at the center of the opening. The base 103 also comprises a passage 105 which extends from the central opening 104 to an edge of the base.

The measuring device 10 comprises a sample holder 5, visible in FIG. 2, in which the post mortem, ex vivo or in vitro skin sample is positioned. The sample holder is mounted on a plate 60 that can move in a vertical direction (Z Z′) normal to the surface of the skin and slidably mounted in a sliding means 61, such as a rail, in a direction parallel to the plane (XY) of the horizontal surface 1. In FIG. 1, the rail extends along the axis (YY′), in the passage 105. Thus, the skin sample is placed at the center of the opening 104 using two positioning adjustments, along the axis (YY′) in a horizontal direction and along the axis (ZZ′) in a vertical direction.

With reference to FIG. 2, the frame 100 comprises attachment means 101, 102 for attaching the base 103 by the suction cup effect on the horizontal surface 1. Any other attachment means can be envisaged. The function of the base 103 is to ensure the stability of the mechanical stress modules 20, 40, 70, 80 during use of the device. The skin sample is placed under traction means. FIG. 2 shows the traction means 30, 50 belonging to the mechanical stress modules 20, 40, respectively.

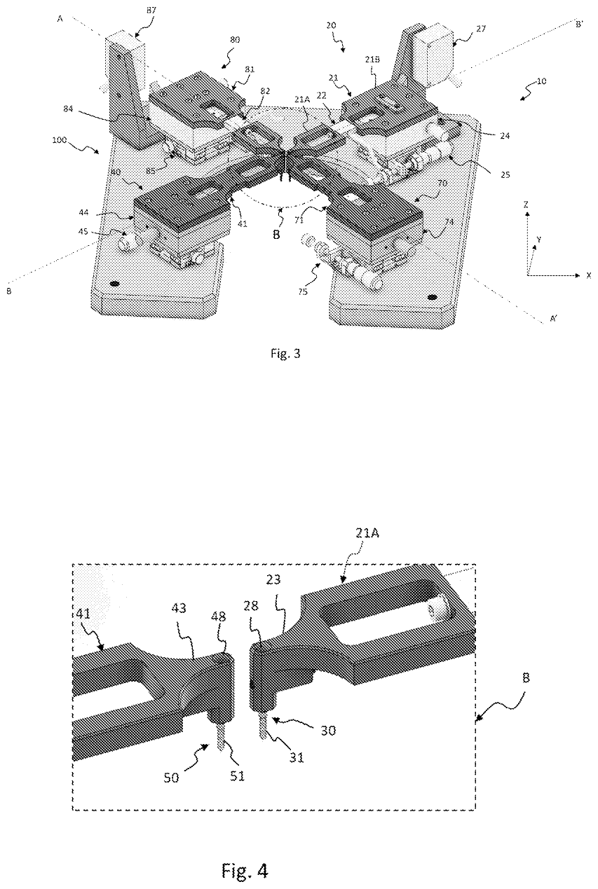

The mechanical stress modules are described in more detail below, with reference to FIG. 3 and to FIG. 4, which is an enlarged view of a region B of FIG. 3.

The first mechanical stress module 20 comprises a first translating arm 21 connected on the one hand to a first traction means 30 and on the other hand to a first displacement means 24. The first traction means 30 comprises an attachment head 31 (visible in FIG. 4) intended to be attached to the skin sample during the traction test. When the first displacement means 24 displaces the translating arm 21, the latter also displaces the traction means 30 along the axis (BB′). The first traction means 30 can induce a deformation of the skin sample by axially displacing a first point of attachment of the skin sample along the axis (BB′).

The second mechanical stress module 40 comprises a second translating arm 41 connected on the one hand to a second traction means 50 and on the other hand to a second displacement means 44. The second traction means 50 comprises an attachment head 51 (visible in FIG. 4) intended to be attached to the skin sample during the traction test. When the second displacement means 44 displaces the translating arm 41, the latter also displaces the traction means 50 along the axis (BB′). The second traction means 50 can induce a deformation of the skin sample by axially displacing a second point of attachment of the sample along the axis (BB′).

The third module 70 and the fourth module 80 are structurally identical to the second module 40 and the first module 20, respectively. Their respective traction means can likewise induce a deformation of the sample by axially displacing two other points of attachment of the sample along the axis (AA′).

The function of the two modules 20, 40 is to exert an opposing tensile force along a common axis (BB′). The function of the two modules 70, 80 is to exert an opposing tensile force along a common axis (AA′).

In the exemplary embodiment illustrated in FIGS. 1 and 3, the mechanical stress modules 20, 40, 70, 80 are arranged around the center of the base 100. More precisely, they are aligned in pairs along the axis (BB′) and the axis (AA′) with the traction means positioned facing one another and positioned substantially at the center of the base 103. Note that the two horizontal translation axes (AA′) and (BB′) are secants at a point located substantially at the center of the opening 104 of the base 103. The skin sample is placed at the center of the opening of the base, under the traction means held by the four mechanical stress modules.

According to one embodiment, the displacement means comprises a piezoelectric nano-positioning table 24, 44, 74, 84. One end of the translating arm 21, 41, 71, 81 is attached on a moving part of the piezoelectric nano-positioning table in order to displace the traction means. The piezoelectric nano-positioning table can control the deformation of the sample by axially displacing the translating arm. In order to exert an opposing tensile force along a common axis, the piezoelectric nano-positioning tables of the aligned mechanical stress modules are likewise aligned in pairs with respect to one another so as to have the same axis of displacement. The piezoelectric nano-positioning table 24 associated with module 20 and the piezoelectric nano-positioning table 44 associated with module 40 have the same axis of displacement (BB′). The piezoelectric nano-positioning table 74 associated with module 70 and the piezoelectric nano-positioning table 84 associated with module 80 have the same axis of displacement (AA′).

According to one embodiment of the invention of the present disclosure, the aligned piezoelectric displacement tables are synchronized so that the displacements of the traction means are synchronized. In this configuration, the tensile force exerted by the opposite displacements of the aligned piezoelectric tables has the same value at any point on the axis of displacement. Thus, a single tensile force sensor is necessary for measuring the tensile force per axis of displacement between the two aligned piezoelectric nano-positioning tables.

According to an advantageous embodiment of the present disclosure, the measuring device comprises a single tensile force sensor 22, 82 per pair of aligned mechanical stress modules, in other words per axis of displacement. In the exemplary embodiment illustrated in FIG. 3, only the first module 20 and the fourth module 80 are equipped, for example, with such a tensile force sensor. More precisely, the force sensor 22, 82 is arranged on the translating arm 21, 81 which comprises a first part 21A, 81A and a second part 21B, 81B. The two parts are connected together by means of a force sensor. The translating arms of the other two modules are formed in a single piece.

The measuring device further comprises one position measurement sensor 27, 87 per pair of aligned mechanical stress modules, in other words per axis of displacement. In the exemplary embodiment illustrated in FIG. 3, only the first module 20 and the fourth module 80 are equipped with such a tensile force sensor 27, 87 which can measure the actual position of the movable part of the piezoelectric displacement table 24, 84 in the frame of reference associated with the frame 100. By way of example, the position measurement sensor is a laser sensor. The laser sensor can thus deduce the actual deformation of the skin sample under the effect of an axial displacement of a point of attachment of the skin sample.

According to one embodiment of the invention, and with reference to FIG. 3, each stress module further comprises a manual micrometric displacement table 25, 45, 75, 85 which can manually adjust the position of the translating arm along one of the translation axes before the start of the traction test. Thus, it is possible to adjust the distance between two attachment heads carried by the translating arms, aligned with one another along the same axis. The piezoelectric nano-positioning table and the micrometric displacement table are arranged with respect to one another, so as to have the same axis of displacement. The piezoelectric nano-positioning table is attached on a movable part of the manual micrometric displacement table. The micrometric displacement table is itself attached to the base 103 of the frame 100. For example, for the two modules 20, 40 aligned with respect to one another, the two manual micrometric displacement tables 25, 45 are likewise aligned with respect to one another so as to have the same axis of displacement. Thus, the four displacement tables 24, 44, 25, 45 are aligned along the axis (BB′).

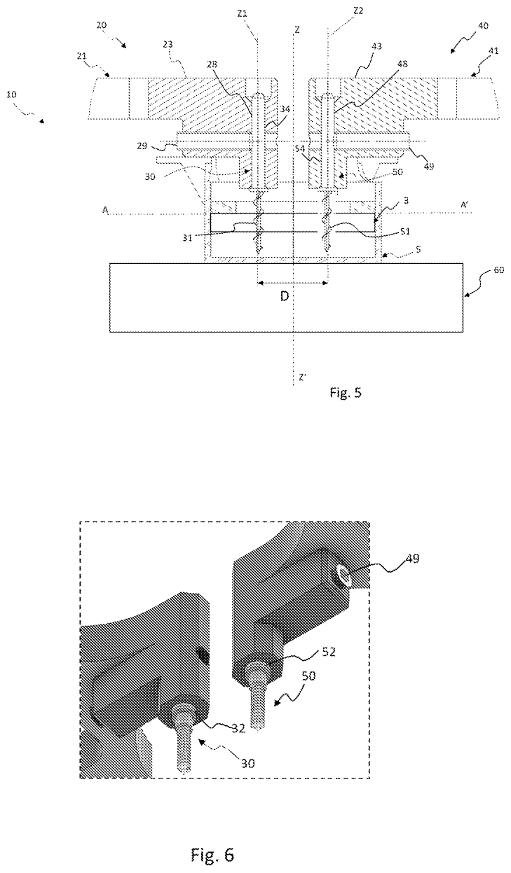

The traction means 30, 50, carried respectively by the aligned modules 20, 40, is described below, with reference to FIGS. 5 and 6.

The traction means 30, 50 comprises a main axis Z1, Z2 oriented in a vertical direction substantially normal to the surface of the skin. The traction means 30, 50 comprises a substantially cylindrical body 34, 54, being provided at one end with an attachment head 31, 51, intended to be attached to the skin 3 during operation of the measuring device 10.

The traction means 30, 50 is attached by mechanical attachment means to the tip 23, 43 of the translating arm 21, 41. A recess 28, 48 is produced in the tip 23, 43. The cylindrical body 34, 54 of the traction means 30, 50 is received in the recess 28, 48 and locked in position using a clamping element 29, 49. The traction means 30, 50 further comprises an annular support surface 32, 52 located at the end of the cylindrical body which is provided with the attachment head. This support surface 32, 52 is capable of abutting against the periphery of the recess 28, 48 when the cylindrical body 34, 54 of the traction means 30, 50 is inserted in the recess. The traction means 30, 50 is removably and interchangeably mounted with respect to the translating arm 21, 41. As illustrated in FIG. 5, once mounted on the tips 23, 43 of the translating arms 21, 41, the two attachment heads 31, 51 are spaced apart at a distance D which can be manually adjusted using the micrometric displacement table 25, 45, which can be seen in FIG. 3.

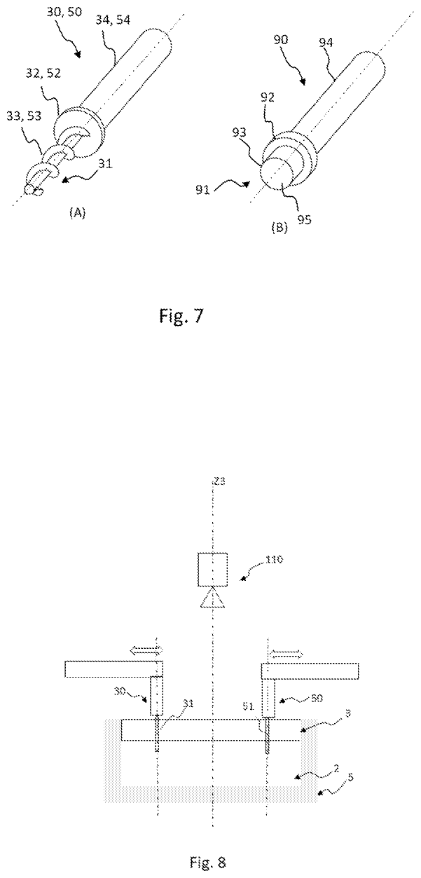

FIG. 7 shows two possible embodiments of the attachment head. According to a first possible embodiment (A), the attachment head is in the form of an attachment rod provided with a thread 33, 53 allowing the rod to be attached in the thickness of the skin by screwing effect.

In the case where this form of attachment head is used, when the test is ended, the operator lowers the sample holder 5 in the vertical direction in order to take the attachment head out of the skin and displaces the sample holder in a direction parallel to the skin on the rail 61 (FIG. 1) so that the sample is no longer in the center of the measuring device.

According to a second embodiment (B), the attachment head 91 is in the form of a substantially cylindrical body 93, the base 95 of which is provided with a layer of adhesive allowing attachment of the attachment head to the surface of the skin. This layer of adhesive can, for example, be a layer of epoxy or any other adhesive suitable for attaching the attachment head to the surface of the skin. In the case where this second embodiment of the attachment head is used in order to obtain a point of attachment on the skin sample, when the traction test is ended, it is necessary to extract the traction means 30, 50 from its recess 28, 48 by unscrewing the clamping screws 29, 49. The traction means 30, 50 are then removed from the measurement region, in other words the center of the base with the attachment heads glued to the surface of the skin sample.

FIG. 8 shows an ex vivo skin sample 3 held in fixed position in a nutritional medium 2. The assembly is contained in the sample holder or receptacle 5. Two attachment heads 31 and 51 are attached by screwing into the thickness of the skin. The two attachment heads are displaced by the displacement means along a common axis in a direction parallel to the surface of the skin. The displacement of each attachment head is represented by a double arrow.

According to one embodiment of the invention, the measuring device also comprises an imaging means 110 configured to observe and record the region of deformation of the surface of the skin caused by the displacement of the attachment heads. By way of example, the imaging means 110 can be a color camera positioned above the surface skin with the optical axis (Z3) oriented in a direction normal to the skin surface, with adjustable magnification, but can also be a more precise microscopy device.

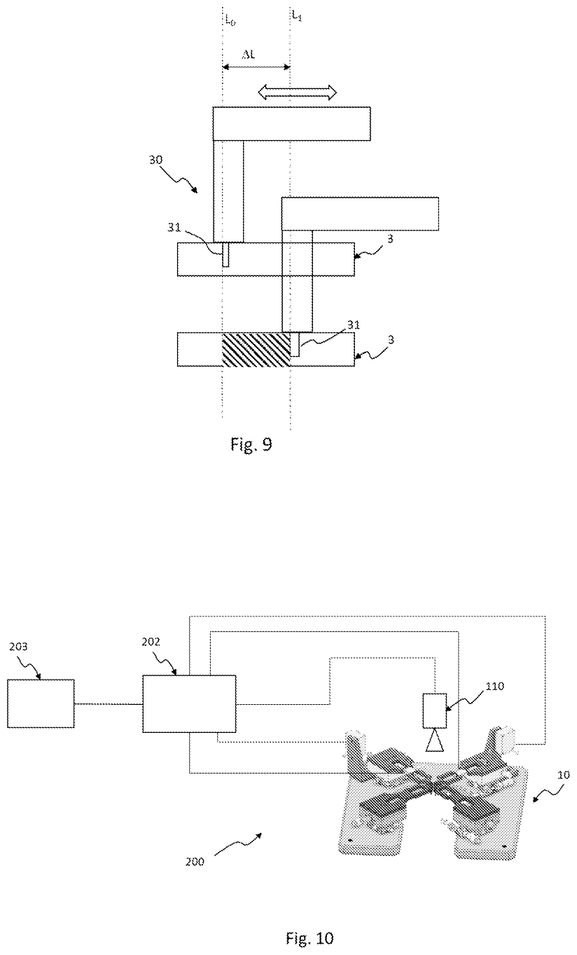

FIG. 9 shows the displacement of one of the attachment heads from an initial position Lo into a position Li in which the attachment head exerts a tensile force on the skin, causing a deformation or an extension of the skin which is represented in FIG. 9 by the distance ΔL traveled by the attachment head. The skin displacement is measured by the laser position sensor 27 (visible in FIG. 3), without short-range contact, projected and aligned on the axis of the translating arm 21 which displaces the attachment head 31.

The position sensors 27, 87 illustrated in FIG. 3 can therefore measure the position of the attachment head with respect to a determined rest or reference position, in particular a rest position of the attachment head as shown in FIG. 9. This position sensor can thus determine the skin deformation caused by the traction applied by the attachment head from the rest position.

FIG. 10 schematically shows a system 200 for measuring mechanical properties, comprising the device 10 for measuring mechanical properties, a control unit 202 of the displacement means and a calculation unit 203.

The system 200 also comprises other means which enable the skin sample to be characterized, for example a radiation scattering system, an ellipsometer or an epi-fluorescence microscope.

The control unit 202 comprises a control program which controls the displacement means 24, 44, 74, 84 of the measuring device 10 in order to displace the translating arms, which displace the corresponding attachment head in translation in the plane of the skin sample.

The tensile force sensors 22, 42, the position measurement sensors 27, 47 and the imaging means 110 are connected to the control unit 202.

According to one embodiment, the control unit is configured to control the displacement means at a stress frequency of between 0.1 mHz and 1 Hz. The shape of the stress frequency can be sinusoidal, triangular or rectangular. More precisely, the control unit 202 is configured to control the displacement of the translating arm in a sinusoidal, triangular or rectangular manner, by varying various parameters such as the stress frequency and the deformation of the sample. The stress frequency can vary between 0.1 mHz and 1 Hz, preferably between 0.1 Hz and 1 Hz and the deformation of the sample can vary between 0.001% and 10%.

The calculation unit 203 is configured to receive signals transmitted by the sensors of the measuring device. The set of signals measured by the sensors of the measuring device is then processed by the calculation unit 203 in order to calculate the mechanical properties of the skin, in particular to track the stress as a function of the deformation.

Controlling the displacement arm in a precise frequency range makes it possible to measure mechanical properties of the skin for large stress times, thus highlighting the effects of viscoelasticity in order to characterize the overall behavior of the skin. Contrary to the stresses at controlled speed for mechanical analysis of a biological tissue usually proposed in the methods of the prior art, the inventors propose a measuring system based on a stress over a range of frequencies, thus making it possible, in a single measurement phase, to determine the evolution of the complex modulus of the skin as a function of various stress frequencies. The measurement of the complex modulus as a function of the stress frequency enables a breakdown into two values: the real part, in phase with the stress signal, which can characterize the elastic properties of the skin and the imaginary part, which can characterize the dissipation properties of the skin.

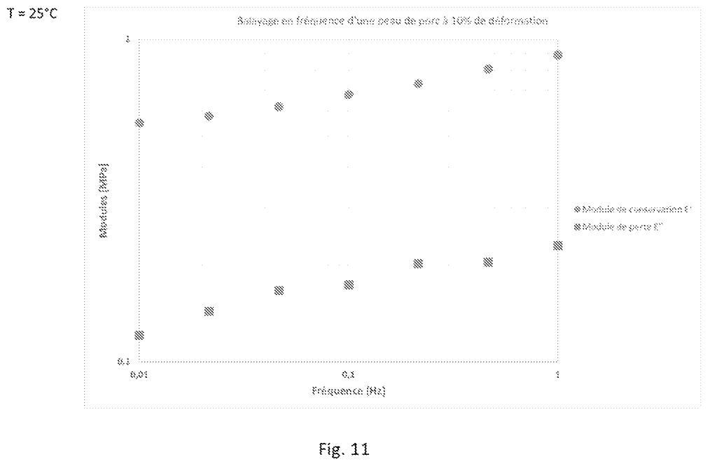

FIG. 11 shows, by way of example, the measurements of the two values at different stress frequency on a pigskin sample at a temperature of 25° C. The round dots correspond to the storage modulus and the square dots correspond to the loss modulus.

### INDUSTRIAL APPLICABILITY

The device 10 of the present disclosure has been designed to enable measuring of the mechanical properties of a human skin sample, ex vivo or in vitro, as a function of a plurality of parameters. The device can impose a deformation on the sample and measure the stress resulting from this deformation. The mechanical stresses are produced with tractions in various directions in the plane of the skin and with various frequencies. Hence, the stress-deformation curves obtained by the device at various frequencies enable elastic, plastic and viscoelastic properties of the skin subjected to a stress to be probed.

Performing the measurements on an ex vivo sample advantageously makes it possible to study the mechanical properties in the region beyond the elastic properties, also referred to as the non-linear region, by subjecting the skin sample to a high deformation.

The device of the present disclosure is particularly suitable for monitoring the evolution over time of the mechanical properties of an ex vivo skin sample, held fixed on a nutritional medium for a duration of several days, and for establishing a link with the potential changes in the mechanical properties of the skin or the structure of the skin induced by an external product. Indeed, holding the skin ex vivo on a nutritional medium enables the skin to keep its mechanical properties as described in document WO2013164436, in contrast to a sample in vitro.

The device makes it possible to continuously monitor, with time over several days, the deformation of the skin and the return of the skin to its equilibrium state, while holding in position attachment points on the same region of the skin throughout the duration of the test, making it possible to obtain precise and reproducible measurements of the mechanical properties of the skin.

The device of the present disclosure can have application in the field of cosmetics and medicine, and any field which treats the human skin.

The device can, for example, based on measurements of mechanical properties, evaluate the changes to healthy skin over time due to the effect of cosmetic products such as moisturizing creams, sun-protection creams or anti-aging creams.

In the medical field, measurements of the mechanical properties make it possible, for example, to monitor the effects of products applied to promote healing or to treat a damaged area. It is also possible to monitor the evolution of the mechanical behavior of an area of the skin damaged by various actions, such as confinement under a dressing or pressure sores under the effect of friction.

The device can also monitor the evolution of the mechanical properties of the skin faced with environmental attacks, such environmental pollution or attack by the sun's rays.

1. A system (200) for measuring the mechanical properties of a skin sample (3) ex vivo or in vitro, comprising a measuring device comprising at least one mechanical stress module (20, 40) capable of applying a tensile force to the skin in a direction parallel to the surface of the skin sample (3), said at least one mechanical stress module (20, 40) comprising:
a traction means (30, 50) which is translatably movable in a direction parallel to the surface of the skin sample (3);
a translating arm (21, 41) connected, on the one hand, to the traction means (30, 50) and, on the other hand, to an axial displacement means;
one end of said traction means being provided with an attachment head (31, 51) capable of being attached to a region of the skin sample (3) so as to cause deformation of the skin sample by axially displacing said region of the skin sample,
a control unit (202) configured to control the displacement means according to a stress frequency of between 0.1 mHz and 1 Hz, and
a calculation unit (203) configured to receive the signals transmitted by the measuring device and calculate the mechanical properties of the skin from said signals.

2. The measurement system as claimed in claim 1, wherein a plurality of mechanical stress modules (20, 40, 70, 80) are arranged around a center of the device and configured to each apply a tensile force along a radial direction parallel to the surface of the skin sample, and the axial displacement means (24, 44, 74, 84) and the translating arms (21, 41, 71, 81) are aligned in pairs so as to displace two traction means along a common displacement axis.

3. The measurement system as claimed in claim 2, wherein the aligned axial displacement means (24, 44, 74, 84) are synchronized so as to simultaneously displace two traction means along the common axis.

4. The measurement system as claimed in one of claims 1 to 3, wherein said axial displacement means comprises a piezoelectric nano-positioning table (24, 44, 74, 84), one end of the translating arm (21, 41, 71, 81) being attached on a moving part of the piezoelectric nano-positioning table (24, 44, 74, 84).

5. The measurement system as claimed in one of claims 1 to 4, wherein each stress module (20, 40, 70, 80) further comprises a manual micrometric displacement table (25, 45, 75, 85) configured to manually adjust the position of the translating arm (21, 41, 71, 81) along one of the axes of displacement.

6. The measurement system as claimed in claims 4 and 5, wherein the piezoelectric nano-positioning table (24, 44, 74, 84) and the micrometric displacement table (25, 45, 75, 85) are arranged with respect to one another so as to have the same axis of displacement.

7. The measurement system as claimed in one of claims 1 to 6, wherein the attachment head (31, 51) is in the form of a rod provided with a thread (33, 53) capable of engaging in the thickness of the skin sample (3) to produce a point of attachment in the skin sample (3).

8. The measurement system as claimed in one of claims 1 to 6, wherein the attachment head (91) is in the form of a straight cylindrical body (93), one of the bases (95) of the straight cylindrical body being provided with a layer of adhesive i to attach the attachment head to the surface of the skin sample.

9. The measurement system as claimed in one of claims 1 to 8, wherein the traction means (30, 50) comprises a cylindrical attachment body (34, 54) intended to be received in a recess (28, 48) produced in one end (23, 43) of the translating arm (21, 41) and locked in position using a clamping element (29, 49).

10. The measurement system as claimed in one of claims 1 to 9, further comprising at least one tensile force sensor (22, 82) capable of measuring the tensile force applied by a traction means.

11. The measurement system as claimed in one of claims 1 to 10, further comprising at least one position measurement sensor (27, 87) capable of measuring the position of a translating arm (21, 81) during its displacement.

12. The measurement system as claimed in one of claims 1 to 11, further comprising at least one imaging means (110) configured to observe the region of deformation of the skin sample caused by the displacement of the attachment heads, the optical axis (Z3) of said imaging means being oriented in a direction normal to the surface of the skin sample.
